(19) United States
(12) Patent Application Publication
(10) Pub. No.: US 2026/0248631 A1
(43) Pub. Date: Aug. 27, 2026

(54) POSTURE TRAINING DEVICE (SHOULDER GIRDLE POSITION CORRECTOR)

(71) Applicants: Ranko COSIC, Chicago, IL (US); Bratislav KOSTIC, Chicago, IL (US)

(72) Inventors: Ranko COSIC, Chicago, IL (US); Bratislav KOSTIC, Chicago, IL (US)

(21) Appl. No.: 19/489,654

(22) PCT Filed: Aug. 15, 2024
(86) PCT No.: PCT/RS2024/000012

§ 371 Date: Dec. 3, 2025

(30) Foreign Application Priority Data
Aug. 31, 2023 (RS) P-2023/0728

(51) Int. Cl. A61F 5/02

(52) U.S. Cl. CPC A61F 5/026

(57) ABSTRACT

The shoulder girdle position corrector includes at least two main assemblies: a supporting structure and a pressure assembly with an adjustment mechanism. The supporting structure includes a pair of bent tubes and an adjustable-length tube assembly, which is positioned horizontally when the corrector is in use. The pressure assembly with an adjustment mechanism includes a pair of pressure elements with adjustment mechanisms and an additional pressure insert.

### CROSS-REFERENCE TO RELATED APPLICATIONS

This application is the U.S. National Phase of PCT/RS2024/000012 Filed Aug. 15, 2024, which claims Priority to P-2023/0728 Filed Aug. 31, 2023, the entire disclosures of which are incorporated by reference herein.

### TECHNICAL FIELD

Shoulder girdle postural correction device is considered part of biomechanics, as they apply biomechanical principles and technological solutions to support and correct shoulder positioning and overall posture.

### TECHNICAL PROBLEM

One of technical problems addressed by the present disclosure is how to implement a corrective device for shoulders that provides support, corrects shoulder positioning, improves overall posture, and reduces symptoms associated with poor posture.

### PRIOR ART

Each user has unique anatomical and corrective needs, making it essential to tailor the shoulder girdle correction device individually while ensuring proper shoulder positioning through continuous pressure applied to the shoulder fold. Traditional shoulder girdle posture correction systems (such as soft fabrics, elastic straps, and fastening buckles) apply continuous force but fail to maintain a fixed shoulder position.

Certain devices, including fabric corsets with cables and rigid corsets made of polymer materials, utilize mechanical systems such as buckles, straps, and braces to provide support and proper spinal alignment. These systems can be adjusted or locked into the desired position to ensure spinal stability.

However, conventional shoulder girdle correction systems using soft fabrics, elastic straps, and fastening buckles exert continuous force without ensuring a fixed shoulder position. The present disclosure, through its specific design and the geometry of its components, maintains a fixed shoulder position during use. The applied shoulder pressure is controlled and can be adjusted throughout the usage period.

### SUMMARY OF THE INVENTION

One of the purposes of the disclosure, a shoulder girdle position corrector, is to provide support and correction for shoulder positioning, improve posture, and reduce symptoms associated with poor posture. These devices are designed to help maintain the proper position of the shoulder girdle, reduce strain on the shoulders and back, and alleviate symptoms such as pain, muscle tension, fatigue, and discomfort caused by poor posture.

Additionally, they assist in correcting forward curvature of the upper spine (kyphosis), which can contribute to better posture and reduced back pain, preventing further deterioration of postural problems. They have potential applications as part of therapy and rehabilitation following injuries or surgeries. The device provides stability, support, and assists in correcting muscles that help maintain proper shoulder positioning.

The goal is to enhance bodily functionality, particularly in the context of postural alignment. Proper shoulder positioning can improve the range of motion, balance, and performance in various activities, including sports and daily tasks. It is important to note that shoulder girdle correction devices are not standalone solutions for postural issues but are part of a comprehensive approach that may include exercises, therapy, habit modifications, and consultation with medical professionals.

One embodiment may this goal through a shoulder girdle position corrector comprising two main assemblies: a supporting structure and a pressure assembly with an adjustment mechanism. The supporting structure includes a pair of interconnected bent metal tubes and an adjustable-length metal tube assembly, positioned horizontally when the corrector is in use. The pressure assembly with an adjustment mechanism consists of a pair of pressure elements with adjustment mechanisms and an additional pressure insert. The shoulder girdle position corrector can be used with the bent metal tubes in a vertical position. Additional features and advantages of the various embodiments will be apparent from the detailed description and the accompanying drawings.

### BRIEF DESCRIPTION OF THE DRAWINGS

An illustrative embodiment will be described in more detail with reference to the attached drawings, in which:

FIG. 1 shows an axonometric view of the disassembled shoulder girdle position corrector;

FIG. 2 shows an axonometric top view of the assembled shoulder girdle position corrector.

### DETAILED DESCRIPTION

The shoulder girdle position corrector according to the invention, which serves as a device for correcting shoulder posture, consists of two main assemblies: a supporting structure and a pressure assembly with an adjustment mechanism.

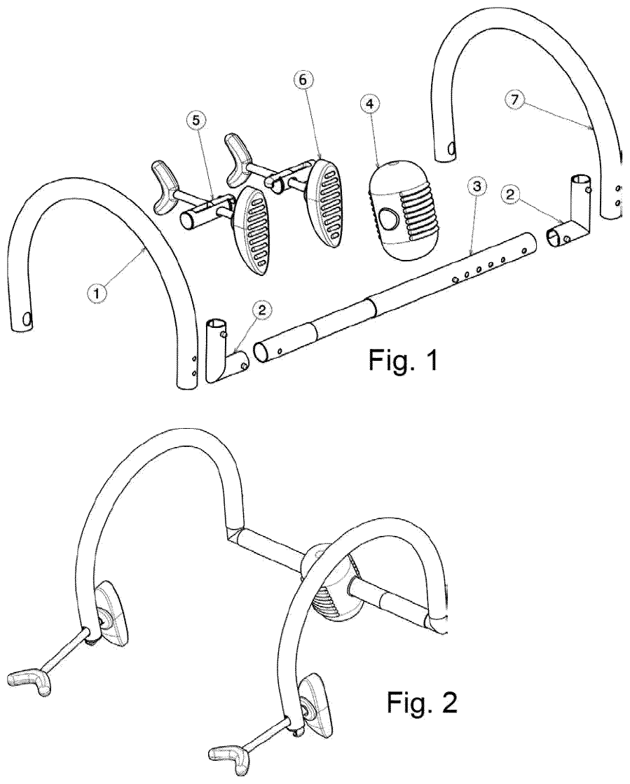

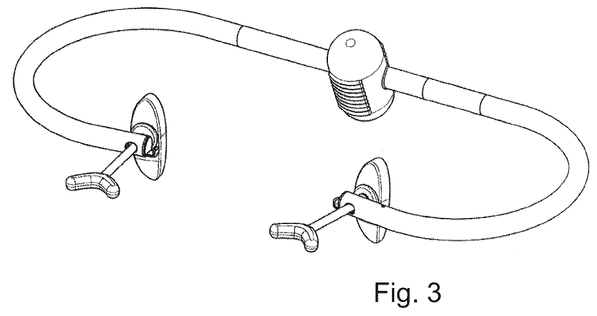

As best seen in FIGS. 1 and 2, the supporting structure includes a pair of bent metal tubes (1, 7) that exert pressure on the shoulders or back. Additionally, the supporting structure contains an adjustable-length metal tube assembly (3), which is positioned horizontally when the corrector is in use. The metal tube assembly (3) consists of metal tubes inserted into one another, allowing length adjustment by extending or retracting them. When fully retracted, the length of the metal tube assembly (3) equals half the average back width of an adult. Although the depicted metal tube assembly (3) in FIGS. 1 and 2 consists of three metal tubes, their number may vary, but there may be at least two to allow length adjustment. At both ends of the metal tube assembly (3), quick-connect fasteners are provided, which will be described in more detail later.

In addition to these components, the supporting structure may include a pair of extensions (2) generally shaped like the letter L. Their perpendicular arms have a smaller diameter than the bent metal tubes (1, 7) and the tubes of the metal tube assembly (3), allowing insertion. Like the metal tube assembly (3), the extensions (2) have quick-connect fasteners at both ends, while the bent metal tubes (1, 7) have such fasteners at only one end. The other end of the bent metal tubes (1, 7) features a through-hole directed towards the first end.

Regarding the aforementioned quick-connect fasteners, they can be implemented in various ways, such as snap-in mechanisms or suitable interference fits between the tubes. A skilled professional in the field could foresee different implementations of these fasteners, so those shown in FIGS. 1-2 are merely non-limiting illustrative examples. In any case, the quick-connect fasteners may enable easy and simple assembly and disassembly of the structural elements they connect, while also being reliable, suitable for mass production, and cost-effective.

FIGS. 1 and 2 illustrate the assembly of the supporting structure when the bent metal tubes (1, 7) are positioned vertically, which is the preferred mode of use. In this configuration, one arm of each extension (2) is inserted into the ends of the bent metal tubes (1, 7), while the other arms are inserted into the ends of the metal tube assembly (3), forming the supporting structure.

The pressure assembly with an adjustment mechanism consists of a pair of pressure elements (6) with adjustment mechanisms (5) and an additional pressure insert (4).

Each pressure element (6) is manufactured through expansion or injection molding of rubber or other synthetic materials resistant to wear, ensuring good grip and comfort. It is made as a single piece with rounded edges for ergonomic support. The surface contour of the pressure element (6) is oval, with one axis of symmetry resembling an egg or an ellipse, which is in direct contact with the body. This shape plays a key role in providing support, comfort, and stability when applying pressure to the shoulder. The front side features different patterns and textures to enhance grip, including patterns resembling parallel, horizontal semi-cylinders with rounded edges. Depending on the model, the surface may be flat or slightly curved to support the natural shape of the shoulder and improve adherence during movement. The rear side of the pressure element (6) is dome-shaped with a circular protrusion that has a central hole for mounting the threaded spindle of the adjustment mechanism (5). The color and appearance of the pressure elements (6) may vary depending on the hardness of the material used. The color coding allows for quick visual identification of hardness and immediate application to the patient. Some pressure elements (6) may be monochrome, while others may have colorful and attractive patterns to draw attention.

Each pressure element (6) has its own adjustment mechanism (5). The mechanism (5) consists of a cylindrical body with a diameter allowing insertion into the bent metal tubes (1, 7). The body of the mechanism (5) features a through-threaded radial hole. When the body of the mechanism (5) is placed inside the bent metal tube (1, 7) so that the hole on one end aligns with the threaded radial hole, a threaded spindle passes through it, entering the central hole on the pressure element (6). The threaded spindle has an enlarged end, such as a butterfly nut, for easier handling. By rotating the threaded spindle via its enlarged end, the user moves the pressure element (6) toward or away from the bent metal tube (1, 7). In the final position, the pressure element (6) can be fully adjusted to rest against the bent metal tube (1, 7).

At the center of the horizontal metal tube assembly (3), there is an insert (4) that applies pressure to the back. This insert (4) can be movable to allow position adjustment based on the desired width of the corrector. It is made of similar materials as the pressure elements (6). The insert (4) has a cylindrical shape with rounded edges, giving it a smooth and rounded appearance. A through-hole is located at the center of the insert (4), but its axis is slightly offset from the longitudinal axis, creating eccentricity. This design allows for adjustment by rotating the cylindrical insert (4) 180 degrees around the metal tube assembly (3) to modify its contact with the user's spine. The cylindrical surface of the insert (4) features two groups of parallel horizontal rounded semi-cylinders on opposite sides, extending along the circumference in an arc, with rounded edges. These groups of semi-cylinders provide more stable positioning of the insert (4) on the user's back.

Referring to FIG. 2, the shoulder girdle position corrector is shown with bent metal tubes (1, 7) in a vertical position.

The device is used by first positioning the pressure elements (6) against the bent metal tubes (1, 7) using the adjustment mechanism (5), creating sufficient space for placing the corrector on the patient's shoulders. The width of the corrector is then adjusted via the horizontal metal tube assembly (3), ensuring that the insert (4) is positioned at the center and aligned with the patient's spine. By tightening the threaded spindle of each adjustment mechanism (5), the pressure elements (6) move horizontally, applying pressure to the shoulders and positioning them in the correct anatomical posture.

In the vertical arrangement of the bent metal tubes (1, 7), as shown in FIG. 2, the patient can move their elbows more freely.

The shoulder girdle position corrector can be used in medical settings as part of therapy and rehabilitation for patients with various conditions, including improper upper torso posture (kyphosis), sports injuries, or post-surgical recovery. Medical professionals, such as orthopedists, physiotherapists, or chiropractors, may recommend and use shoulder position correctors as part of comprehensive treatment; they can also be used preventively to avoid such issues and improve posture.

Shoulder position correctors can also be used in everyday life to support and maintain proper posture. Individuals prone to poor posture or those with a sedentary lifestyle can use these devices to help maintain correct shoulder alignment and support proper posture.

Athletes and recreational practitioners engaged in activities requiring good posture, such as gymnastics, ballet, tennis, and golf, may use shoulder position correctors to improve posture and reduce the risk of injury. These devices can provide support during training and competition, helping athletes maintain proper shoulder positioning and enhance their sports performance.

In all these applications, it is important to follow the manufacturer's instructions and consult with a medical professional to determine the best use and proper adjustment of the shoulder position corrector.

1. A shoulder girdle position corrector including two main assemblies, comprising; a supporting structure and a pressure assembly with an adjustment mechanism, wherein the supporting structure includes a pair of interconnected bent tubes joined by an adjustable-length tube assembly positioned along a back of a user during use, while the pressure assembly with the adjustment mechanism includes of a pair of pressure elements each with adjustment mechanisms configured to move the pressure elements towards and away from the user during use.

2. The shoulder girdle position corrector of claim 1, wherein the tubes of the tube assembly are fully retracted into each other, and the length of the tube assembly is equal to half the average back width of an adult.

3. The shoulder girdle position corrector of claim 1, wherein the supporting structure also includes a pair of extensions that allow for connecting of the bent tubes to the adjustable horizontal assembly.

4. The shoulder girdle position corrector of claim 3, wherein the tube assembly has connecting fasteners at both ends, while the bent tubes have such fasteners only at one end, with the other end featuring a through-hole directed toward the first end.

5. The shoulder girdle position corrector of claim 3, wherein the extensions have connecting points at both ends.

6. The shoulder girdle position corrector of claim 1, wherein the pressure assembly with the adjustment mechanism also includes an additional pressure insert.

7. The shoulder girdle position corrector of claim 6, wherein each pressure element is configured to directly contact the body, wherein the pressure element includes a front surface including ridges to improve adherence to the body, and the front surface is either flat or contoured to support the natural shoulder shape and improve fit, while the back side of the pressure element is dome-shaped with a circular protrusion containing a central hole.

8. The shoulder girdle position corrector of claims 6, wherein the adjustment mechanism includes a cylindrical body with a diameter allowing insertion into the bent tubes, wherein the mechanism body includes a threaded radial through-hole, so that when the mechanism body is placed inside the bent tube so that a hole at one end aligns with the threaded radial through-hole, a threaded spindle is inserted through it, entering the central hole of the pressure element, while the threaded spindle has an enlarged end.

9. The shoulder girdle position corrector of claim 6, wherein the insertis movably positioned on the horizontal tube assembly and consists of a cylindrical shape with rounded edges, through-hole at its center, possibly with its axis slightly offset from the longitudinal axis to achieve eccentricity, and the cylindrical surface of the insert featuring two groups of parallel horizontal rounded semi-cylinders on opposite sides, arranged in an arc along the circumference, with rounded edges.

10. The shoulder girdle position corrector of claim 6, the pressure elements and insert are manufactured through expansion or injection molding of rubber or other materials resistant to wear.

11. A shoulder girdle position corrector comprising:
a supporting structure that includes at a pair of bent tubes each configured to engage with a corresponding shoulder of a user when worn, wherein the pair of bent tubes are connected via a substantially horizontal tube configured to adjust in size via either expanding or contracting;
an insert located at the substantially horizontal tube ; and
a pair of pressure assemblies that include an adjustment mechanism, wherein the pair of pressure assemblies are located at the pair of bent tubes opposite, wherein the pressure assembly with the adjustment mechanism includes one or more pressure elements configured to adjust an amount of pressure applied at a front portion of the corresponding shoulder of the user in response to activation of the adjustment mechanism either moving towards or away from the corresponding shoulder of the user when worn.

12. The should girdle position corrector of claim 11, wherein the adjustment mechanism includes a threaded spindle that corresponds to a threaded hole, wherein the adjustment mechanism is configured to move toward or away from the pair of interconnected bent tubes in response to rotation of the adjustment mechanism.

13. The shoulder girdle of claim 11, wherein the pair of bent tubes are joined at the substantially horizontal tube by one or more angular elements, wherein the one or more angular elements are configured to adjust a height of the pair of bent tubes.

14. The shoulder girdle of claim 11, wherein the insert is configured to be movable along the substantially horizontal tube via an aperture located in the insert that encompasses the substantially horizontal tube and further configured to rotate along a same as the substantially horizontal tube.

15. The shoulder girdle of claim 11, where in the insert is cylindrical and made of a same material as the pressure assembly.

16. A position corrector comprising:
a first bent portion configured to engage with a first shoulder of a user;
a second bent portion configured to engage with a second shoulder of the user;
a tube including a first end and a second end, wherein the first end is connected to the first bent portion and the second end is connected to the second bent portion;
an insert located at the tube and configured to align with a middle portion of a back of the user when worn, wherein the insert encompasses a portion of the tube; and
a first pressure assembly located at the first bent portion and includes a first adjustment mechanism configured to move the first pressure assembly either towards or away the user when the position corrector is worn;
a second pressure assembly located at the second bent portion and includes a first adjustment mechanism configured to move the first pressure assembly either towards or away the user when the position corrector is worn.

17. The position corrector of claim 16, wherein the insert is substantially cylindrical in shape and aligned vertically off-axis from the tube.

18. The position corrector of claim 16, wherein the insert, the first pressure assembly, and the second pressure assembly are all a same material.

19. The position corrector of claim 16, wherein the insert is configured to be rotatable along the tube and moveable horizontally along the tube.

20. The position corrector of claim 16, wherein the first bent portion, the second portion, the tube, the first pressure assembly, and the second pressure assembly are all detachable components.
